(19) United States
(12) Patent Application Publication
(10) Pub. No.: US 2010/0331566 A1
(43) Pub. Date: Dec. 30, 2010

(54) SYNTHETIC METHODS AND COMPOSITIONS

(76) Inventors: Leslie S. Jimenez, New Brunswick, NJ (US); Ahalya Ramanathan, New Brunswick, NJ (US)

Correspondence Address: VIKSNINS HARRIS & PADYS PLLP, P.O. BOX 111098, ST. PAUL, MN, 55111-1098, US

(21) Appl. No.: 12/823,937

(22) Filed: Jun. 25, 2010

Related U.S. Application Data
(60) Provisional application No. 61/220,939, filed on Jun. 26, 2009.

(51) Int. Cl. C07C 253/30; C07C 201/12; C07C 231/12; C07C 41/24; C07C 37/00; C07C 45/65; C07C 209/74; C07C 205/06; C07C 233/07; C07C 43/205; C07C 39/06; C07C 49/563; C07C 211/52; C07C 255/50

(52) U.S. Cl. 558/411; 562/434; 564/218; 568/658; 568/782; 568/315; 564/412; 568/940; 568/781; 568/308; 564/442

(57) ABSTRACT

The invention provides synthetic methods that utilize bromo or chloro substituents as blocking groups during the functionalization of aromatic rings, as well as compounds that are prepared from such methods.

### PRIORITY OF INVENTION

This application claims priority from U.S. Provisional Application No. 61/220,939, filed on 26 Jun. 2009. The entire content of this provisional application is hereby incorporated by reference herein.

### GOVERNMENT FUNDING

The invention described herein was made with government support under Grant Number CHE 0316199 awarded by the National Science Foundation. The United States Government has certain rights in the invention.

### FIELD OF THE INVENTION

The present invention is directed to synthetic methods for use in the substitution of aromatic rings and the compositions produced therefrom. More particularly, the methods of the present invention involve the use of bromo or chloro substituents as blocking groups during functionalization of aromatic rings.

### BACKGROUND

Regioselective synthesis of ortho-substituted benzenes can be achieved through the use of a blocking group at the para position. Functionalization at one or both ortho positions, followed by removal of the blocking group yields the ortho-substituted aromatic compound. Sulfonic acids have long been used as para-blocking groups (see Sen, A. B.; Kulkarni, Y. D. J. Indian Chem. Soc. 1956, 33, 326-328; Sakellarios, E.; Jatrides, D. Berichte der Deutschen Chemischen Gesellschaft [Abteilung] B: Abhandlungen 1925, 58B, 2286-2288). However this method is not compatible with acid-sensitive functional groups. Current methods of ortho-substitution are limited to particular types of substrates and/or substituents (see Moutrille, C.; Zard, S. Z. Tetrahedron Lett. 2004, 45, 4631-4634; Zhu, L.; Zhang, M. J. Org. Chem. 2004, 69, 7371-7374; J. Am. Chem. Soc. 1978, 100, 4842-4847; Heteroatom Chemistry 1998, 9, 549-551; Aust. J. Chem. 1988, 41, 69-80).

There are a wide variety of methods available to reduce aryl bromides (see Pinder, A. R. Synthesis 1980, 425-452). The reversibility of electrophilic bromination on activated arenes (e.g. anilines, phenols, anisoles) means that the equilibrium can be shifted towards the debrominated product in the presence of a bromine scavenger such as sodium sulfite, toluene, or aniline (see Choi, H. Y.; Chi, D. Y. J. Am. Chem. Soc. 2001, 123, 9202-9203; Adimurthy, S.; Ramachandraiah, G.; Bedekar, A. V. Tetrahedron Lett. 2003, 44, 6391-92; Adimurthy, S.; Ramachandraiah, G. Tetrahedron Lett. 2004, 45, 5251-52; Tashiro, M.; Watanabe, H.; Tsuge, O. Organic Prep. Procedures Intl. 1975, 7, 43-46; and Effenberger, F. Angew. Chem. Int. Ed. 2002, 41, 1699-1700).

Aryl bromides can also be reduced by lithiation, followed by quenching with water (see van Zijl, P. C. M. Et al., J. Am. Chem. Soc. 1986, 108, 1415-1418; Jensen, J., et al., J. Org. Chem. 2002, 67, 6008-6014; Pyne, S. G.; Boche, G. J. Org. Chem. 1989, 54, 2663-2667). Heterogeneous Pd/C-catalyzed reduction with formate salts gives good yields of dehalogenated products (see Cortese, N. A.; Heck, R. F. J. Org. Chem. 1977, 42, 3491-3494; Wiener, H.; Blum, J.; Sasson, Y. J. Org. Chem. 1991, 56, 6145-6148). Aryl bromo groups have been selectively reduced in the presence of chloro substituents using zinc in a basic solution, a homogenous Pd(0) catalyst and polymethylhydrosiloxane as the hydrogen donor, or 10% Pd/C under a hydrogen atmosphere (see Tashiro, M.; Fukata, G. J. Org. Chem. 1977, 42, 835-838; Pi-Bar, I.; Buchman, O. J. Org. Chem. 1986, 51, 734-736; and Shankar, R. B.; Pews, R. G. J. Heterocyclic Chem. 1993, 30, 169-172; and Takahashi, M. et al., Tetrahedron 1999, 55, 5295-5302).

Conditions for reducing aryl chlorides are generally more vigorous than conditions for reducing aryl bromides. Photolysis of chlorophenols in an alcohol solvent leads to the dehalogenated products (see Manet, I. Et al., Chem. Eur. J. 2005, 11, 140-151). Chloroanilines have been reduced with a formate hydrogen donor and a Pd catalyst (see Pews, R. G.; Hunter, J. E.; Wehmeyer, J. M. Tetrahedron 1993, 49, 4809-4820). Sodium hypophosphite and sodium borohydride have also been used as hydrogen donors to reduce chlorobenzenes (see Boyer, S. K. et al., J. Org. Chem. 1985, 50, 3408-3411; and Lassova, L. et al., J. Mclr. Cat. A: Chemical 1999, 144, 397-403). Hydrogenation with a polymer-supported palladium catalyst shows some degree of regioselectivity in the reduction of polychlorinated benzenes (see Zhang, Y.; Liao, S.; Xu, Y. Tetrahedron Lett. 1994, 35, 4599-4602). Hydrodehalogenation with Pd/C and dihydrogen in a multiphase system rapidly reduces polychlorinated benzenes completely (see Marques, C. A.; Selva, M.; Tundo, P. J. Org. Chem. 1993, 58, 5256-5260). Triethylamine has been shown to selectively enhance the activity of Pd/C catalysts towards reduction of aryl chlorides relative to other sensitive functional groups (see Monguchi, Y. et al., Tetrahedron 2006, 62, 7926-7933). A nitro group was reduced under these conditions.

Hydrogen gas is attractive as a hydrogen donor due to its relatively low price and ease of use, making it the reactant of choice for industrial applications (see Weissermel, K.; Arpe, H.-J. Industrial Organic Chemistry, ed. 2, VCH: Weinheim 1993, 23-27; and Mukhopadhyay, S. et al., Tetrahedron 1999, 55, 14763-14768).

### SUMMARY OF THE INVENTION

It has been determined that bromo and chloro substituents can serve as excellent blocking groups on aromatic rings. With a halo substituent para to an ortho,para-directing group, the ortho positions on the aromatic rings can be functionalized and then the halo group can be removed under neutral conditions using catalytic hydrogenation. As expected, bromides are reduced using shorter reaction times and lesser amounts of catalyst than chlorides. Bromides can be selectively reduced in the presence of nitro, chloro, cyano, keto, and carboxylic acid groups.

In one embodiment the invention provides a method for preparing a 1,2 substituted aromatic compound comprising reducing a corresponding 1,2-substituted aromatic compound that also comprises a bromo or chloro group to provide the 1,2-substituted aromatic compound.

In another embodiment the invention provides a method of preparing a 1,2-substituted aromatic ring in an aromatic compound comprising reducing a corresponding aromatic compound having a 1,2-substituted aromatic ring that also comprises a bromo or chloro group to provide the 1,2-substituted aromatic ring.

In another embodiment the invention provides a method of preparing a 1,2-substituted aromatic ring in an aromatic compound having a 1-substituted aromatic ring comprising: substituting a bromo or chloro group onto the 1-substituted aromatic ring 3 positions away from the substitution at position 1; substituting a group on the aromatic ring 1 position away from the substitution at position 1 and 2 positions away from the bromo or chloro group; and reducing the compound to provide the 1,2-substituted aromatic ring in the aromatic compound.

Advantageously, the methods of the present invention are generally applicable and enable functional groups to be introduced in patterns not otherwise readily obtainable. Accordingly, the present invention is also directed to compositions produced from the methods of the present invention. The methods of the present invention allow for the removal of the blocking groups in the presence of sensitive groups such as nitro or cyano groups.

### DETAILED DESCRIPTION OF THE INVENTION

The following definitions are used, unless otherwise described. Alkyl, alkoxy, alkanoyl, etc. denote both straight and branched groups; but reference to an individual radical such as propyl embraces only the straight chain radical, a branched chain isomer such as isopropyl being specifically referred to. Aryl denotes a phenyl radical or a fused bicyclic or tricyclic carbocyclic radical having about nine to fourteen ring atoms in which at least one ring is aromatic. Heteroaryl encompasses a radical of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an fused bicyclic or tricyclic heterocycle of about eight to fourteen ring atoms comprising one to four heteroatoms each selected from the group consisting of non-peroxide oxygen, sulfur, and N(X) in which at least one ring is aromatic.

Scheme 1 illustrates a representative reaction sequence for aromatic substitution according to the method of the invention.

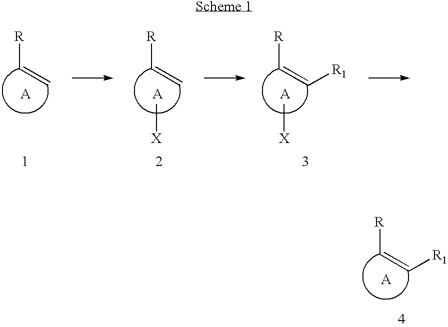

In Scheme 1, “A” represents an aromatic ring and X represents a chloro or bromo group. Suitable aromatic rings include aryl and heteroaryl groups as defined wherein the ring bearing R and X is aromatic. It is to be understood that “A” can be further substituted or that “A” can form part of a larger organic molecule, provided the groups substituted on “A” or the remainder of the organic molecule comprising “A” do not interfere with the reaction sequence shown in Scheme 1. Since “A” can be further substituted or can form part of a larger molecule, it is to be understood that the term “1,2-substituted” as used herein describes compounds that are “substituted on adjacent carbons of an aromatic ring;” the term is not limited to compounds wherein the priority rules of organic nomenclature would dictate that the compound is substituted at the 1-priority position and the 2-priority position.

The compound of formula 1 can be halogenated using any suitable method to provide the bromo or chloro compound 2 (see for example R. T. Morrison and R. N. Boyd, Organic Chemistry 4^{th} ed., Allyn and Bacon, Inc, 1983, 577, 594, 605, 968, 975, and 993-995; and Jerry March, Advanced Organic Chemistry 4^{th} ed., John Wiley and Sons, 1992, 531-534). Alkylation of 2 using any suitable conditions provides the 1,2-substituted aromatic ring that also comprises a bromo or chloro group 3. For example, compound 2 can be alkylated by electrophylic aromatic substitution to provide compound 3 (see for example R. T. Morrison and R. N. Boyd, Organic Chemistry 4^{th} ed., Allyn and Bacon, Inc, 1983, 593-623; and Jerry March, Advanced Organic Chemistry 4^{th} ed., John Wiley and Sons, 1992, 501-568). Reduction of 3 provides the 1,2 disubstituted aromatic compound 4. Compound 3 can be reduced using any suitable conditions (see for example Choi, H. Y.; Chi, D. Y. J. Am. Chem. Soc. 2001, 123, 9202-9203; Adimurthy, S.; Ramachandraiah, G.; Bedekar, A. V. Tetrahedron Lett. 2003, 44, 6391-92; Adimurthy, S.; Ramachandraiah, G. Tetrahedron Lett. 2004, 45, 5251-52; Tashiro, M.; Watanabe, H.; Tsuge, O. Organic Prep. Procedures Intl. 1975, 7, 43-46; Effenberger, F. Angew. Chem. Int. Ed. 2002, 41, 1699-1700; van Zijl, P. C. M. Et al., J. Am. Chem. Soc. 1986, 108, 1415-1418; Jensen, J., et al., J. Org. Chem. 2002, 67, 6008-6014; Pyne, S. G.; Boche, G. J. Org. Chem. 1989, 54, 2663-2667; Cortese, N. A.; Heck, R. F. J. Org. Chem. 1977, 42, 3491-3494; Wiener, H.; Blum, J.; Sasson, Y. J. Org. Chem. 1991, 56, 6145-6148; Tashiro, M.; Fukata, G. J. Org. Chem. 1977, 42, 835-838; Pi-Bar, I.; Buchman, O. J. Org. Chem. 1986, 51, 734-736; Shankar, R. B.; Pews, R. G. J. Heterocyclic Chem. 1993, 30, 169-172; and Takahashi, M. et al., Tetrahedron 1999, 55, 5295-5302; Manet, I. Et al., Chem. Eur. J. 2005, 11, 140-151; Pews, R. G.; Hunter, J. E.; Wehmeyer, J. M. Tetrahedron 1993, 49, 4809-4820; Boyer, S. K. et al., J. Org. Chem. 1985, 50, 3408-3411; and Lassova, L. et al., J. Mclr. Cat. A: Chemical 1999, 144, 397-403; Zhang, Y.; Liao, S.; Xu, Y. Tetrahedron Lett. 1994, 35, 4599-4602; Marques, C. A.; Selva, M.; Tundo, P. J. Org. Chem. 1993, 58, 5256-5260; Monguchi, Y. et al., Tetrahedron 2006, 62, 7926-7933; Weissermel, K.; Arpe, H.-J. Industrial Organic Chemistry, ed. 2, VCH: Weinheim 1993, 23-27; and Mukhopadhyay, S. et al., Tetrahedron 1999, 55, 14763-14768). In one embodiment of the invention, the compound 3 is reduced by catalytic hydrogenation, for example, using palladium on carbon as a catalyst. As illustrated in the Examples below, the conversion of compound 3 to compound 4 can conveniently be carried out using 10% Pd/C in methanol at room temperature in the presence of a suitable acid scavenger (e.g. sodium bicarbonate).

In one embodiment of the invention “A” is a phenyl ring. Scheme 2 further illustrates this embodiment of the invention.

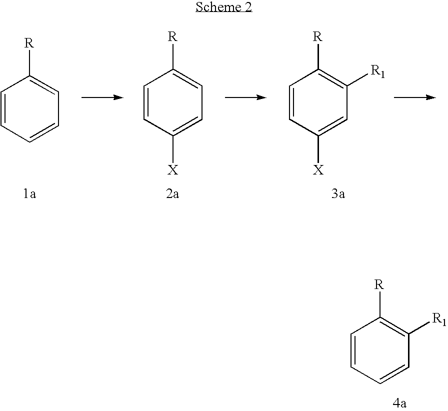

In the Examples below, a series of readily available bromo- and chlorobenzenes was reduced using catalytic hydrogenation to illustrate the principle of aryl halides as blocking groups. For bromides, using 0.6 mol % of 10 wt. % Pd/C in methanol was sufficient to remove the bromide at room temperature within an hour. Three equivalents of sodium bicarbonate were added to neutralize the hydrogen bromide by-product. In the presence of other easily reducible substituents, the reaction was carefully monitored by thin layer chromatography (tlc) to determine when to stop the reaction. In the case of 4-bromo-2-nitrobenzoic acid (entry 8, Table 1), increasing the catalyst amount slightly, removed the bromide more rapidly and minimized reduction of the nitro group. Reduction of chlorides 3 was easily achieved within an hour by increasing the mol % of catalyst used (entries 4 and 5). Although the reactions appeared to have been converted almost completely into product by thin layer chromatography, the isolated yields of the more volatile products (entries 3, 6 and 9) were low due to the small scale of the reactions. Yields, reaction times and mole percents of the palladium catalyst are given in Table 1 for various benzene substrates.

Specific values and embodiments identified herein are for illustration only; they do not exclude other defined values or other values within defined ranges for the radicals and substituents.

In one embodiment of the invention the 1,2 substituted aromatic compound is substituted at the 1-position with an ortho,para-directing group.

In one embodiment of the invention the 1,2-substituted aromatic compound that also comprises a bromo or chloro group is a compound of formula 3a:

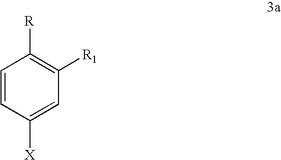

wherein R and R₁ are each suitable organic groups and X is chloro or bromo.

In one embodiment of the invention R is an ortho,para-directing group.

In one embodiment of the invention the ortho,para-directing group is selected from fluoro, (C₁-C₆)alkyl, (C₁-C₆)alkoxy, hydroxy, and NRₐR_{b}; wherein Rₐ and R_{b} are each independently selected from H, (C₁-C₆)alkyl, and (C₁-C₆)alkanoyl.

In one embodiment of the invention R is selected from fluoro, (C₁-C₆)alkyl, (C₁-C₆)alkoxy, hydroxy, NRₐR_{b}, cyano, carboxy, and nitro; Rₐ and R_{b} are each independently selected from H, (C₁-C₆)alkyl, and (C₁-C₆)alkanoyl; and X is chloro or bromo.

In one embodiment of the invention R is selected from fluoro, chloro, (C₁-C₆)alkyl, (C₁-C₆)alkoxy, hydroxy, NRₐR_{b}, cyano, carboxy, and nitro; Rₐ and R_{b} are each independently selected from H, (C₁-C₆)alkyl, and (C₁-C₆)alkanoyl; and X is bromo.

In one embodiment of the invention the bromo or chloro 1,2-substituted aromatic compound is reduced by catalytic hydrogenation.

In one embodiment of the invention the catalytic hydrogenation is carried out in the presence of a palladium catalyst.

In one embodiment of the invention the palladium catalyst comprises palladium on carbon.

In one embodiment of the invention the 1,2-substituted aromatic compound that also comprises a bromo or chloro group is prepared by converting a corresponding 1-substituted aromatic compound that also comprises a bromo or chloro group to the 1,2-substituted aromatic compound that also comprises a bromo or chloro group.

In one embodiment of the invention the 1-substituted aromatic compound that also comprises a bromo or chloro group is converted to the 1,2-substituted aromatic compound that also comprises a bromo or chloro group by alkylating the corresponding 1-substituted aromatic compound that also comprises a bromo or chloro group to provide the 1,2-substituted aromatic compound that also comprises a bromo or chloro group.

In one embodiment of the invention the 1-substituted aromatic compound that also comprises a bromo or chloro group is alkylated by electrophylic substitution to provide the 1,2-substituted aromatic compound that also comprises a bromo or chloro group.

In one embodiment of the invention a 1-substituted aromatic compound that also comprises a bromo or chloro group of formula 2a:

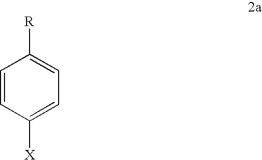

wherein R is a suitable group and X is chloro or bromo; is converted to a 1,2-substituted aromatic compound compound that also comprises a bromo or chloro group of formula 3a:

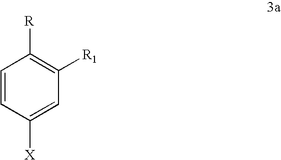

wherein R₁ is a suitable group; and X is chloro or bromo.

In one embodiment of the invention R₁ is selected from (C₁-C₆)alkyl, fluoro, nitro, and (C₁-C₆)alkanoyl.

In one embodiment of the invention R₁ is selected from (C₁-C₆)alkyl, fluoro, chloro, nitro, and (C₁-C₆)alkanoyl; and X is bromo.

In one embodiment of the invention the 1-substituted aromatic compound that also comprises a bromo or chloro group is prepared by brominating or chlorinating a corresponding 1-substituted aromatic compound to provide the bromo or chloro 1-substituted aromatic compound.

In one embodiment of the invention the 1-substituted aromatic compound is a compound of formula 1a:

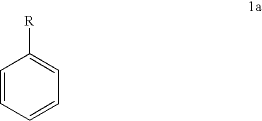

wherein R is a suitable group.

The present invention is described more fully by way of the following non-limiting examples.

Melting points (uncorrected) were measured on a Thomas Hoover apparatus. 1H and 13C NMR spectra were carried out on Varian VNMRS 300, 400 and 500 MHz spectrometers. Methanol was distilled over calcium hydride.

### EXAMPLES

### Example 1

### 2-Nitrobenzoic acid

To a dry 3-necked, 50-mL flask under a N₂ atmosphere, was added, 4-bromo-2-nitrobenzoic acid (0.12 g, 0.49 mmol), 10 wt. % Pd/C (4.3 mg, 0.82 mol %), NaHCO₃ (0.133 g, 3.2 mmol) and 1.8 ml of freshly distilled, dry CH3OH. With a balloon on the center neck, the flask was filled with H₂ with three vacuum-fill cycles, and the reaction mixture was vigorously stirred at room temperature for the indicated time (Table 1). After 25 minutes, the solution was filtered through Celite, and the solvent was removed in vacuo. The residue was purified by flash chromatography on silica gel using 1:4 CH₃OH/CH₂Cl₂ as the eluant. 2-Nitrobenzoic acid was isolated as light yellow crystals (75 mg, 0.45 mmol, 92%). Mp: 145-148° C. (lit. mp: 146-148° C21). 1H NMR (300 MHz, CDCl3+DMSO-d6): δ 7.51 (t, J=7.7 Hz, 1H), 7.60 (t, J=7.4 Hz, 1H), 7.71 (d, J=8.1 Hz, 1H), 7.77 (d, J=7.2 Hz, 1H). 13C NMR (300 MHz, CDCl3+DMSO-d 6): δ 127.9, 134.6, 134.9, 137.2, 138.7, 153.6, 173.7.

### Example 2

### 2-Fluoroacetanilide

This compound was prepared as described above for 2-nitrobenzoic acid. From 94 mg of 4-bromo-2-fluoroacetanilide (entry 1) was isolated 2-fluoroacetanilide (62 mg, 100%) as a white powder. Mp: 78-80° C. (lit. mp: 78-79° C23). 1H NMR (500 MHz, CDCl₃): δ 2.21 (s, 3H), 6.92-7.14 (m, 3H), 7.57 (s, NH), 8.26 (t, J=7.8 Hz, 1H). ¹³C NMR (500 MHz, CDCl3): δ 24.6, 114.7 (d, JCF=75.5 Hz), 121.9, 124.3 (d, JCF=31.5 Hz), 124.6 (d, JCF=15.0 Hz), 126.4 (d, JCF=41.0 Hz), 152.4 (d, JCF=964.5 Hz), 168.4.

### Example 3

### 2-Methylacetanilide

This compound was prepared as described above for 2-nitrobenzoic acid. From 230 mg of 4-bromo-2-methylacetanilide (entry 2) was isolated 2-methylacetanilide (149 mg, 99%) as a white solid. Mp: 111-113° C. (lit. mp: 110-112° C24). 1H NMR (300 MHz, CDCl₃): δ 2.04 (s, 3H), 2.14 (s, 3H), 6.98-7.14 (m, 3H), 7.45 (d, J=7.5 Hz, 1H), 7.84 (br s, 1H). 13C NMR (300 MHz, CDCl3): δ 18.1, 23.9, 124.8, 125.9, 126.6, 130.7, 131.3, 135.9, 169.5.

### Example 4

### 2-Methylanisole

This compound was prepared as described above for 2-nitrobenzoic acid. From 272 mg of 4-bromo-2-methylanisole (entry 3) was isolated 2-methylanisole (49.2 mg, 30%) as a pale yellow liquid (flash chromatography with 19:1 petroleum ether:EtOAc). 1H NMR (300 MHz, CDCl₃): δ 2.52 (s, 3H), 3.86 (s, 3H), 6.82-6.93 (m, 2H), 7.12-7.25 (m, 2H). 13C NMR (300 MHz, CDCl3): δ 16.4, 55.5, 110.2, 120.5, 126.8, 127.0, 130.8, 159.5.

### Example 5

### 2-Isopropyl-5-methylphenol

This compound was prepared as described above for 2-nitrobenzoic acid. From 200 mg of 4-chloro-5-methyl-2-isopropylphenol (entry 4) was isolated 5-methyl-2-isopropylphenol (130 mg, 80%) as white crystals. Mp: 48-51° C. (lit. mp: 46-47° C.). 1H NMR (500 MHz, CDCl3): δ 1.33 (d, J=7 Hz, 6H), 2.35 (s, 3H), 3.34 (hep, 1H), 5.08 (s, OH), 6.63 (s, 1H), 6.83 (d, 7.5 Hz, 1H), 7.18 (d, 8 Hz, 1H). 13C NMR (500 MHz, CDCl3): δ 21.1, 23.0, 27.0, 116.4, 122.0, 126.6, 131.8, 136.9, 152.8.

### Example 6

### 2′-Hydroxyacetophenone

This compound was prepared as described above for 2-nitrobenzoic acid. From 164.5 mg of 5′-chloro-2′-hydroxyacetophenone (entry 5) was isolated 2′-hydroxyacetophenone (122.5 mg, 93%) as a light yellow liquid. 1H NMR (400 MHz, CDCl₃): δ 2.59 (s, 3H), 6.87 (dt, J=7.6, 1 Hz, 1H), 6.94 (dd, J=8.4, 1.2 Hz, 1H), 7.43 (dt, J=7.8, 1.4 Hz, 1H), 7.70 (dd, J=8, 1.6 Hz, 1H). 13C NMR (400 MHz, CDCl3): δ 26.8, 118.6, 119.2, 119.9, 131.0, 136.7, 162.6, 204.8.

### Example 7

### 2-Chloroaniline

This compound was prepared as described above for 2-nitrobenzoic acid. From 124 mg of 4-bromo-2-chloroaniline (entry 6) was isolated 2-chloroaniline (30 mg, 39%) as a yellow liquid (flash chromatography with 9:1 petroleum ether:EtOAc). 1H NMR (500 MHz, CDCl₃): δ 4.01 (s, NH2, 2H), 6.71 (dt, J=7.8, 1.5 Hz, 1H), 6.77 (dd, J=8, 1.3 Hz, 1H), 7.08 (dt, J=7.5, 1.3 Hz, 1H), 7.26 (dd, J=8, 1.5 Hz, 1H). 13C NMR (500 MHz, CDCl3): δ 115.9, 119.0, 119.3, 127.6, 129.4, 142.9.

### Example 8

### 2-Chlorobenzonitrile

This compound was prepared as described above for 2-nitrobenzoic acid. From 169 mg of 4-bromo-2-chlorobenzonitrile (entry 7) was isolated 2-chlorobenzonitrile (80.5 mg, 75%) as a pale yellow solid (flash chromatography with 9:1 petroleum ether:EtOAc). Mp: 44-46° C. (lit. mp: 44-45° C.). 1H NMR (300 MHz, CDCl₃): δ 7.31 (dt, 7.2, 2.7 Hz, 1H), 7.41-7.52 (m, 2H), 7.59 (dd, J=7.8, 0.6 Hz, 1H). 13C NMR (300 MHz, CDCl3): δ 113.6, 116.2, 127.4, 130.3, 134.2, 134.3, 137.1.

### Example 9

### 2-Nitrotoluene

This compound was prepared as described above for 2-nitrobenzoic acid. From 124 mg of 2-bromo-6-nitrotoluene (entry 9) was isolated 2-nitrotoluene (41 mg, 52%) as a yellow liquid (flash chromatography with 9:1 petroleum ether:EtOAc). 1H NMR (400 MHz, CDCl₃): δ 2.59 (s, 3H), 7.23-7.29 (m, 2H), 7.41 (dt, J=7.6, 1.4 Hz, 1H), 7.88 (dd, J=8.8, 1.2 Hz, 1H). 13C NMR (400 MHz, CDCl3): δ 20.6, 124.8, 127.1, 133.0, 133.2, 133.8, 149.5.

Table 1 shows the product and yield obtained under specified conditions of mole % catalyst and reaction time for nine reactants according to the methods of the present invention.

TABLE 1

| | | | Pd/C | Time | Yield |
|---|---|---|---|---|---|
| Example | Reactant | Product | (mol %) | (min) | (%) |
| 1 | 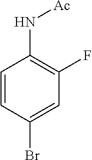 | 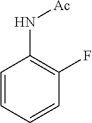 | 0.60 | 40 | 100 |
| 2 | 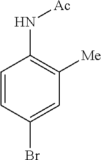 | 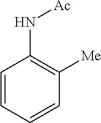 | 0.60 | 50 | 99 |
| 3 | 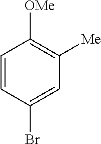 | 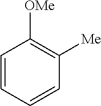 | 0.60 | 40 | 30 |
| 4 | 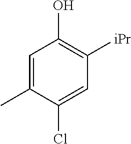 | 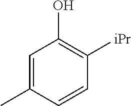 | 1.5 | 60 | 80 |
| 5 | 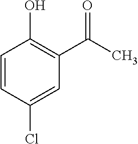 | 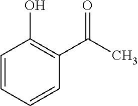 | 1.5 | 50 | 93 |
| 6 | 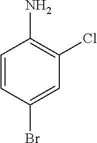 | 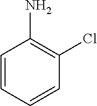 | 0.60 | 22 | 39 |
| 7 | 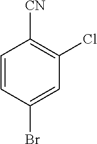 | 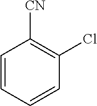 | 0.60 | 45 | 75 |
| 8 | 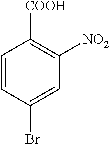 | 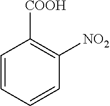 | 0.82 | 25 | 92 |
| 9 | 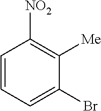 | 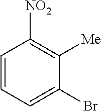 | 0.60 | 35 | 52 |

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method for preparing a 1,2 substituted aromatic compound comprising reducing a corresponding 1,2-substituted aromatic compound that also comprises a bromo or chloro group to provide the 1,2-substituted aromatic compound.

2. The method of claim 1 wherein the aromatic compound is a phenyl ring.

3. The method of claim 1 wherein the 1,2 substituted aromatic compound is substituted at the 1-position with an ortho,para-directing group.

4. The method of claim 1 wherein the 1,2-substituted aromatic compound that also comprises a bromo or chloro group is a compound of formula 3a: 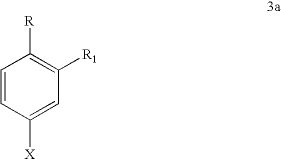
wherein R and R₁ are each suitable organic groups and X is chloro or bromo.

5. The method of claim 4 wherein R is an ortho,para-directing group.

6. The method of claim 3 wherein the ortho,para-directing group is selected from fluoro, (C₁-C₆)alkyl, (C₁-C₆)alkoxy, hydroxy, and NRₐR_{b}; wherein Rₐ and R_{b} are each independently selected from H, (C₁-C₆)alkyl, and (C₁-C₆)alkanoyl.

7. The method of claim 4 wherein R is selected from fluoro, (C₁-C₆)alkyl, (C₁-C₆)alkoxy, hydroxy, NRₐR_{b}, cyano, carboxy, and nitro; Rₐ and R_{b} are each independently selected from H, (C₁-C₆)alkyl, and (C₁-C₆)alkanoyl; and X is chloro or bromo.

8. The method of claim 4 wherein R is selected from fluoro, chloro, (C₁-C₆)alkyl, (C₁-C₆)alkoxy, hydroxy, NRₐR_{b}, cyano, carboxy, and nitro; Rₐ and R_{b} are each independently selected from H, (C₁-C₆)alkyl, and (C₁-C₆)alkanoyl; and X is bromo.

9. The method of claim 1 wherein the 1,2-substituted aromatic compound that also comprises a bromo or chloro group is reduced by catalytic hydrogenation.

10. The method of claim 9 wherein the catalytic hydrogenation is carried out in the presence of a palladium catalyst.

11. The method of claim 10 wherein the palladium catalyst comprises palladium on carbon.

12. The method of claim 1 further comprising preparing the 1,2-substituted aromatic compound that also comprises a bromo or chloro group by converting a corresponding 1-substituted aromatic compound that also comprises a bromo or chloro group to the 1,2-substituted aromatic compound that also comprises a bromo or chloro group.

13. The method of claim 12 wherein the 1-substituted aromatic compound that also comprises a bromo or chloro group is converted to the 1,2-substituted aromatic compound that also comprises a bromo or chloro group by alkylating the corresponding 1-substituted aromatic compound that also comprises a bromo or chloro group to provide the 1,2-substituted aromatic compound that also comprises a bromo or chloro group.

14. The method of claim 13 wherein the 1-substituted aromatic compound that also comprises a bromo or chloro group is alkylated by electrophilic substitution to provide the 1,2-substituted aromatic compound that also comprises a bromo or chloro group.

15. The method of claim 12 wherein a 1-substituted aromatic compound that also comprises a bromo or chloro group of formula 2a: 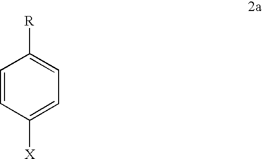
wherein R is a suitable group and X is chloro or bromo; is converted to a 1,2-substituted aromatic compound compound that also comprises a bromo or chloro group of formula 3a: 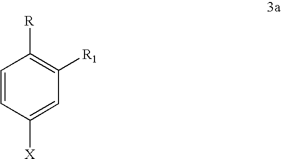
wherein R₁ is a suitable group; and X is chloro or bromo.

16. The method of claim 15 wherein R is an ortho,para-directing group.

17. The method of claim 16 wherein the ortho,para-directing group is selected from fluoro, (C₁-C₆)alkyl, (C₁-C₆)alkoxy, hydroxy, and NRₐR_{b}; wherein Rₐ and R_{b} are each independently selected from H, (C₁-C₆)alkyl, and (C₁-C₆)alkanoyl.

18. The method of claim 15 wherein R is selected from fluoro, (C₁-C₆)alkyl, (C₁-C₆)alkoxy, hydroxy, NRₐR_{b}, cyano, carboxy, and nitro; Rₐ and R_{b}, are each independently selected from H, (C₁-C₆)alkyl, and (C₁-C₆)alkanoyl; and X is chloro or bromo.

19. The method of claim 15 wherein R is selected from fluoro, chloro, (C₁-C₆)alkyl, (C₁-C₆)alkoxy, hydroxy, NRₐR_{b}, cyano, carboxy, and nitro; Rₐ and R_{b} are each independently selected from H, (C₁-C₆)alkyl, and (C₁-C₆)alkanoyl; and X is bromo.

20. The method of claim 15 wherein R₁ is selected from (C₁-C₆)alkyl, fluoro, nitro, and (C₁-C₆)alkanoyl.

21. The method of claim 15 wherein R₁ is selected from (C₁-C₆)alkyl, fluoro, chloro, nitro, and (C₁-C₆)alkanoyl; and X is bromo.

22. The method of claim 15 further comprising preparing the 1-substituted aromatic compound that also comprises a bromo or chloro group by brominating or chlorinating a corresponding 1-substituted aromatic compound to provide the bromo or chloro 1-substituted aromatic compound.

23. The method of claim 22 wherein the corresponding 1-substituted aromatic compound is a compound of formula 1a: 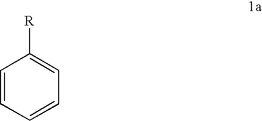
wherein R is a suitable group.

24. The method of claim 23 wherein R is an ortho,para-directing group.

25. The method of claim 24 wherein the ortho,para-directing group is selected from fluoro, (C₁-C₆)alkyl, (C₁-C₆)alkoxy, hydroxy, and NRₐR_{b}; wherein Rₐ and R_{b} are each independently selected from H, (C₁-C₆)alkyl, and (C₁-C₆)alkanoyl.

26. The method of claim 23 wherein the compound of formula 1 is brominated or chlorinated and R is selected from fluoro, (C₁-C₆)alkyl, (C₁-C₆)alkoxy, hydroxy, NRₐR_{b}, cyano, carboxy, and nitro; Rₐ and R_{b} are each independently selected from H, (C₁-C₆)alkyl, and (C₁-C₆)alkanoyl.

27. The method of claim 23 wherein the compound of formula 1 is brominated and R is selected from fluoro, chloro, (C₁-C₆)alkyl, (C₁-C₆)alkoxy, hydroxy, NRₐR_{b}, cyano, carboxy, and nitro; Rₐ and R_{b} are each independently selected from H, (C₁-C₆)alkyl, and (C₁-C₆)alkanoyl.

28. A 1,2-substituted aromatic compound prepared by the process of claim 1.

29. A method of preparing a 1,2-substituted aromatic ring in an aromatic compound comprising reducing a corresponding aromatic compound having a 1,2-substituted aromatic ring that also comprises a bromo or chloro group to provide the 1,2-substituted aromatic ring.

30. The method of claim 29 wherein the bromo or chloro group is three positions away on the aromatic ring from the substitution at position 1 and two positions away from the substitution at position 2.

31. The method of claim 29, wherein the aromatic ring is a six member ring.

32. A method of preparing a 1,2-substituted aromatic ring in an aromatic compound having a 1-substituted aromatic ring comprising:
substituting a bromo or chloro group onto the 1-substituted aromatic ring 3 positions away from the substitution at position 1;
substituting a group on the aromatic ring 1 position away from the substitution at position 1 and 2 positions away from the bromo or chloro group; and
reducing the compound to provide the 1,2-substituted aromatic ring in the aromatic compound.
